(19) United States
(12) Patent Application Publication
(10) Pub. No.: US 2020/0046223 A1
(43) Pub. Date: Feb. 13, 2020

(54) HOME APPLIANCE AND CLOUD SERVER PERFORMING HEALTHCARE FUNCTION USING ARTIFICIAL INTELLIGENCE

(71) Applicant: LG ELECTRONICS INC., Seoul (KR)

(72) Inventor: Cheolyeon LEE, Seoul (KR)

(73) Assignee: LG ELECTRONICS INC., Seoul (KR)

(21) Appl. No.: 16/525,295

(22) Filed: Jul. 29, 2019

(30) Foreign Application Priority Data
Aug. 7, 2018 (KR) 10-2018-0091971

(51) Int. Cl. A61B 5/00; G06N 20/00; A61B 5/0205; A61B 5/0408; A61B 5/1172

(52) U.S. Cl. CPC A61B 5/0022; G06N 20/00; A61B 5/0205; A61B 5/02427; A61B 5/746; A61B 5/1172; A61B 5/7264; A61B 5/0408

(57) ABSTRACT

Provided are a home appliance and a cloud server that perform a healthcare function using artificial intelligence. According to the present disclosure, the home appliance and the cloud server includes a controller that compares an average of health information value for a predetermined period of time and current the health information value of the user based on health information value including the electrocardiogram measured by an electrocardiogram measuring unit and the heart rate measured by a heart rate measuring unit. As a result, the controller may determine an emergency situation. In this case, the controller may generate a message with respect to the emergency situation and may transmit the generated message to a user terminal registered in advance.

### CROSS-REFERENCE TO RELATED APPLICATION

Pursuant to 35 U.S.C. § 119(a), this application claims the benefit of earlier filing date and right of priority to Korean Patent Application No. 10-2018-0091971, filed on Aug. 7, 2018, the contents of which are hereby incorporated by reference herein in its entirety.

### BACKGROUND

### 1. Field of the Invention

A home appliance and a cloud server that perform a healthcare function using artificial intelligence are disclosed herein.

### 2. Description of Related Art

Home appliances used in a predetermined space such as home or offices, and the like may perform their own functions and operations. Examples of a home appliance may include a washing machine, a dryer, an air conditioner, a robot cleaner, an air purifier, a refrigerator, an oven range, a water purifier, and the like.

Among them, the refrigerator maintains a low temperature of various types of stored goods or cooling a temperature of various types of stored goods to a low temperature. In other words, the refrigerator has been used to keep food fresh for a long period of time.

Recently, various kinds of additional functions of refrigerators are provided to users by combining the refrigerator with a large-screen display and various types of communication modules. For example, the refrigerator may operate to maximize energy efficiency of the refrigerator by analyzing the number of opening the door and data with respect to an increase in temperature inside of the refrigerator.

However, in the related art, the refrigerator may not provide a function for determining a health state of the user and determining normality or abnormality of health of the user based on information on the health state of the user.

Further, in the related art, the refrigerator does not take any action even when the refrigerator may detect a sudden change in a use pattern of the user. For example, when a user, as senior citizen who lives alone, does not use the refrigerator for a predetermined period of time, the health abnormality is likely to occur to the user. However, in this case, the related art refrigerator may not take any action.

Therefore, in the modern society in which aging is rapidly progressed and one person household is rapidly increasing, the need for a function for automatically transmitting a call message to a predetermined person is increased when the use pattern of the user is analyzed and it is determined that a problem occurs to the user based on the use pattern of the user.

### SUMMARY OF THE INVENTION

The present disclosure provides a home appliance and a cloud server that may measure health information value through a sensor provided on a handle of a home appliance and periodically checking health abnormality of a user based on the health information value.

The present disclosure further provides a home appliance and a cloud server that may determine a possibility of occurrence of an emergency situation using machine learning with a pattern of door opening measured by the home appliance as a learning factor.

The present disclosure further provides a home appliance and a cloud server that may determine the emergency situation based on a use pattern of a user with respect to the home appliance and the health information value of the user and may transmit a call message to a terminal registered in advance during occurrence of the emergency situation.

The objects of the present disclosure are not limited to the above-mentioned objects, and other objects and advantages of the present disclosure which are not mentioned may be understood by the following description and more clearly understood by the implementations of the present disclosure. It will also be readily apparent that the objects and the advantages of the present disclosure may be implemented by means appended in claims and a combination thereof.

According to the present disclosure, the home appliance and the cloud server may include a controller that compares an average of the health information value during a predetermined period of time with current health information value of the user based on the health information value including the electrocardiogram measured by an electrocardiogram measuring unit and the heart rate measured by a heart rate measuring unit, so that the controller may determine the emergency situation. In this case, the controller may generate a message with respect to the emergency situation and may transmit the generated message to a user terminal registered in advance.

In addition, according to the present disclosure, the home appliance and the cloud server include a learning unit that receives a previously stored pattern of opening the door as a learning factor and outputs a matching rate of the previously stored pattern of opening the door with a current pattern of opening the door as an output thereof. When the matching rate of the previously stored pattern of opening the door with the current pattern of opening the door is less than a predetermined reference matching rate, the message with respect to the emergency situation may be generated and transmitted to the user terminal registered in advance.

In addition, according to the present disclosure, the home appliance and the cloud server include a controller that determines an emergency situation based on a use pattern of a user with respect to the home appliance and health information value of the user. Accordingly, the present disclosure may enable transmitting a call message to a terminal registered in advance when the emergency situation occurs.

According to the present disclosure, the home appliance and the cloud server may reduce efforts of the user to visit a hospital for healthcare by measuring the health information value of the user by the sensor provided on a handle of the home appliance. In addition, the user may detect a symptom of disease early by periodically managing the normality or abnormality of the health of the user.

In addition, according to the present disclosure, the home appliance and the cloud server may determine whether the emergency situation occurs by comparing a general pattern of opening the door of a user with a recent pattern of opening the door of a user. At this time, the present disclosure may enable improving accuracy of determining the emergency situation using machine learning. Therefore, the present disclosure may enable quickly responding to the accident occurring to a user as one person household.

Further, according to the present disclosure, the home appliance and the cloud server may improve the accuracy of determining whether the emergency situation occurs based on both the use pattern of the user with respect to the home appliance and health information value of the user. As a result, reliability of the user with respect to a product may be improved. Further, brand awareness of the product may also be improved.

The effects of the present disclosure are not limited to the effects described above, and those skilled in the art of the present disclosure may readily understand various effects obtained by the present disclosure based on the specific description of the disclosure.

### BRIEF DESCRIPTION OF THE DRAWINGS

FIG. 1 is a perspective view of a home appliance according to an implementation of the present disclosure.

FIG. 2 is a partially enlarged view of a handle of the home appliance of FIG. 1.

FIG. 3 shows positions and types of sensor arranged in sensing areas of FIG. 1.

FIG. 4 is a schematic view of a configuration of a heart rate measuring unit arranged in the sensing area of FIG. 1.

FIG. 5 is a block diagram of a home appliance according to an implementation of the present disclosure.

FIG. 6 is a schematic view of a home appliance and a cloud server according to another implementation of the present disclosure.

FIG. 7 is a block diagram of relations between the home appliance and the cloud server of FIG. 6.

FIG. 8 shows a configuration of a learning unit according to some implementations of the present disclosure.

FIG. 9 is a flowchart of a method of operating a home appliance according to some implementations of the present disclosure.

FIG. 10 shows a method for determining normality or abnormality of a pattern of opening the door in S140 of FIG. 9.

### DETAILED DESCRIPTION OF EXEMPLARY EMBODIMENTS

The advantages and features and a method for achieving them of the present disclosure will become apparent with reference to the implementations described below in detail with reference to the accompanying drawings. The present disclosure may, however, be implemented in many different manners and should not be construed as being limited to the implementations set forth herein. Rather, these implementations are provided so that this disclosure will be thorough and complete, and will fully convey the scope of the disclosure to those skilled in the art to which the present disclosure pertains, and the disclosure is only defined by the scope of claims. Like reference numerals indicate like elements throughout the disclosure.

Unless defined otherwise, all terms (including technical and scientific terms) used herein may be used in a sense commonly understood by one of ordinary skill in the art to which the present disclosure pertains. Further, commonly used terms, which are defined in a dictionary, are not ideally or excessively interpreted unless explicitly and particularly defined otherwise.

Further, with respect to the implementation of the present disclosure, for convenience of explanation, the present disclosure may be described by subdividing components; however, these components may be implemented within one apparatus or module, or a component may be implemented by being divided into a plurality of apparatuses or modules.

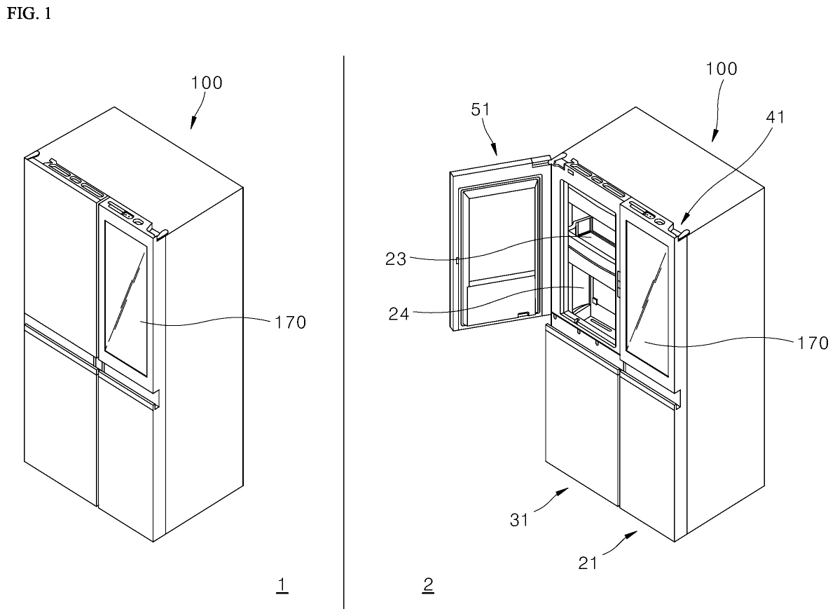

Hereinafter, a home appliance and a cloud server that perform a healthcare function according to some implementation of the present disclosure will be described with reference to FIGS. 1 to 10.

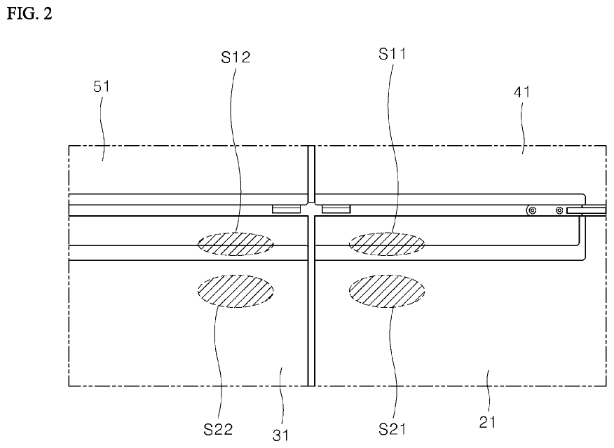

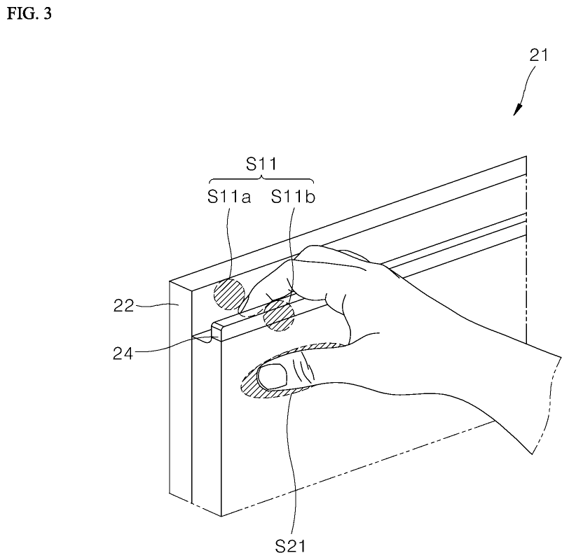

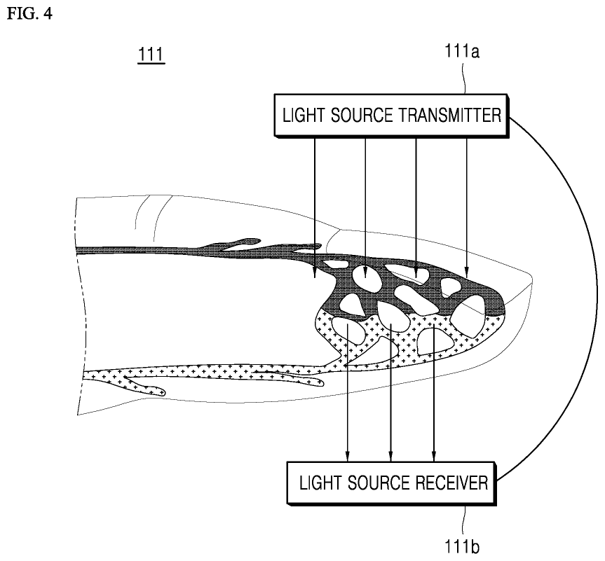

FIG. 1 is a perspective view of a home appliance according to an implementation of the present disclosure. FIG. 2 is a partially enlarged view of a handle of the home appliance of FIG. 1. FIG. 3 shows positions and types of a sensor arranged in a sensing area of FIG. 1. FIG. 4 is a schematic view of a configuration of a heart rate measuring unit arranged in the sensing area of FIG. 1.

A home appliance 100 may include a refrigerator, a washing machine, a dryer, an oven range, and the like. At this time, the home appliance 100 may include various types of home appliances that have a handle that a user may grip. However, this is only a portion of implementations of the various types of home appliances 100, and the present disclosure is not limited thereto.

In the present disclosure, for convenience of explanation, the refrigerator will be described.

Referring to FIG. 1, according to an implementation of the present disclosure, in a home appliance 100 (that is, a refrigerator), a first shape 1 shows an appearance of a home appliance 100 when a door is closed. A second shape 2 shows an appearance of the home appliance 100 when the door is open.

The home appliance 100 may include a plurality of doors 21, 31, 41, and 51. In addition, the home appliance 100 may have a plurality of storage spaces divided by the plurality of doors 21, 31, 41, and 51.

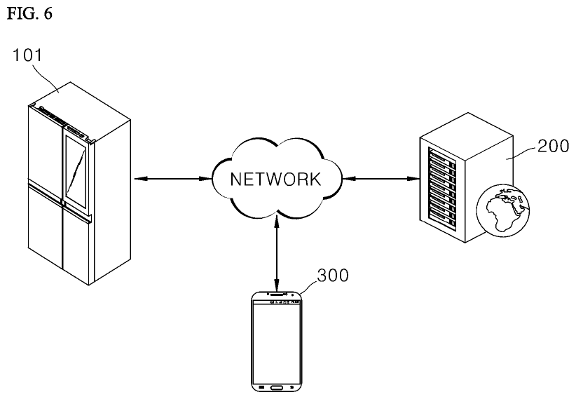

Each of doors 21, 31, 41 and 51 may include a door sensor (119 of FIG. 6) that senses a state of the doors 21, 31, 41 and 51 (that is, an open state or a close state) and a temperature sensor (117 of FIG. 6) that senses a temperature of each storage space of the home appliance.

Further, each of doors 21, 31, 41, and 51 may further include a heart rate measuring unit (111 of FIG. 6) that may measure a heart rate of a user, a fingerprint recognizer (i.e. fingerprint sensor) that may measure a fingerprint of the user (113 of FIG. 6), and an electrocardiogram measuring unit (115 of FIG. 6) that may measure an electrocardiogram of the user.

Referring to FIGS. 2 and 3, the heart rate measuring unit 111 may be provided inside of a handle formed on the doors 21 and 31.

For example, the heart rate measuring unit 111 may be arranged in a first area S11 located inside of the handle of the first door 21. At this time, the first door 21 includes a base plate 22 that forms a body and a handle 24 that is formed at one side of the base plate 22 so that the user may grip the handle.

A first area S11 has a first sub-area S11a arranged at one side of the base plate 22 and a second sub-area S11b arranged inside of the handle 24. At this time, the first sub-area S11a and the second sub-area S11b may be spaced apart from each other. For example, the first sub-area S11a and the second sub-area S11b may face each other.

Referring to FIG. 4, the heart rate measuring unit 111 may include a light source transmitter 111a and a light source receiver 111b.

The light source transmitter 111a may emit light of a predetermined magnitude. The types of light received from the light source transmitter 111a may be infrared rays, visible light, ultraviolet rays, and the like. The light source transmitter 111a may be variously modified and implemented.

The light source receiver 111b receives the light from the light source transmitter 111a, which is emitted from the light source transmitter 111a. The light source receiver 111b generates electric signals that have different kinds of magnitude from one another depending on an amount of received light. At this time, the generated electric signal may be used to measure the heart rate of the user.

Specifically, the light source transmitter 111a transmits the light to an artery of the user. Then, the light source receiver 111b may sense the light transmitted to a vein through the artery of the user, using a photo detector. At this time, the light source receiver 111b may calculate the amount of received light.

The light source transmitter 111a may be disposed in the first sub-area S11a and the light source receiver 111b may be disposed on the second sub-area S11b. Therefore, when the user places his/her finger on a handle 24 of a first door 21, the light source transmitter 111a transmits the light on an upper surface of the finger. At this time, the light source receiver 111b is adjacent to a lower surface of the finger, and senses the light transmitted to the finger.

For reference, this is only an example, and the positions of the light source transmitter 111a and the light receiver 111b may be variously modified and implemented.

Referring back to FIGS. 2 and 3, a heart rate measuring unit 111 may be arranged in the first area S11 of the first door 21 and the second area S12 of the second door 31, respectively. At this time, a structure of the heart rate measuring unit 111 formed on the first area S11 and the second area S12 may be the same as the structure described with reference to FIG. 4.

The fingerprint recognizer 113 may recognize the fingerprint of the user. A structure and operation principle of the fingerprint recognizer 113 are disclosed in detail in the prior art documents, and a detailed description thereof is omitted. For example, the fingerprint recognizer 113 may recognize a fingerprint pattern of the user using a capacitive method. At this time, the skilled person in the art may easily implement the fingerprint recognizer 113 with reference to known documents.

The fingerprint recognizer 113 may be arranged in a third area S21 and a fourth area S22. That is, the fingerprint recognizer 113 may be arranged on an outer surface of the first door 21 and on an outer surface of the second door 31. In addition, the fingerprint recognizer 113 may be arranged in at least one of the third area S21 and the fourth area S22.

Therefore, when the user places his or her hand on the handle 24, the fingerprint of the thumb of the user may naturally contact the fingerprint recognizer 113. However, this is merely one example, and the position of the fingerprint recognizer 113 may be variously modified and implemented.

The electrocardiogram measuring unit 115 may measure the electrocardiogram of the user. The structure and operation principle of the electrocardiogram measuring unit 115 are disclosed in detail in the conventional known art, and a detailed description will be omitted. The skilled person in the art may easily implement the electrocardiogram measuring unit 115 with reference to the known document.

The electrocardiogram measuring unit 115 may be arranged in the third area S21 and the fourth area S22 like the fingerprint recognizer 113. That is, the electrocardiogram measuring unit 115 may be arranged on an outer surface of the first door 21 and on an outer surface of the second door 31.

At this time, the electrocardiogram measuring unit 115 may be arranged in both the third area S21 and the fourth area S22. That is, the electrocardiogram of the user may be measured by a pair of the first electrocardiogram measuring unit 115a arranged in the third area S21 and the second electrocardiogram measuring unit 115 arranged in the fourth area S22.

The user may measure his or her waveform of electrocardiogram by placing one hand on the first electrocardiograph measuring unit 115a and placing the other hand on the second electrocardiogram measuring unit 115b. At this time, the first electrocardiogram measuring unit 115a and the second electrocardiogram measuring unit 115b may be installed on different doors from each other. However, this is only one example, and the position of the electrocardiogram measuring unit 115 may be variously modified and implemented.

In summary, in the first door 21, the heart rate measuring unit 111 may be arranged in the first area S11 and the fingerprint recognizer 113 and the electrocardiogram measuring unit 115 may be arranged in the third area S21. At this time, the fingerprint recognizer 113 and the electrocardiogram measuring unit 115 may be manufactured as an integrated module.

For reference, a piezoelectric sensor may be further provided in the first area S11 and the third area S21. The piezoelectric sensor may sense whether the hand of the user contacts the first door 21. Then, the controller 140 described below may provide each sensor 110 (that is, the above-mentioned heart rate measuring unit 111, the fingerprint recognizer 113, and the electrocardiogram measuring unit 115) with power only when a predetermined pressure is sensed by the piezoelectric sensor, thereby minimizing amount of power used by each sensor 110.

For reference, the home appliance 100 may further include an interface 170 that displays state information of the home appliance 100 or shows an inside of the home appliance 100. The user may control the operation of the home appliance 100 through the interface 170. In addition, the user may access a network or control other electronic devices through the interface 170.

The interface 170 may be arranged on a front surface of a specific door (for example, 31). However, a position and a shape of the interface 170 may be variously modified and implemented.

Hereinafter, the components included in the home appliance 100 according to an implementation of the present disclosure will be described in detail.

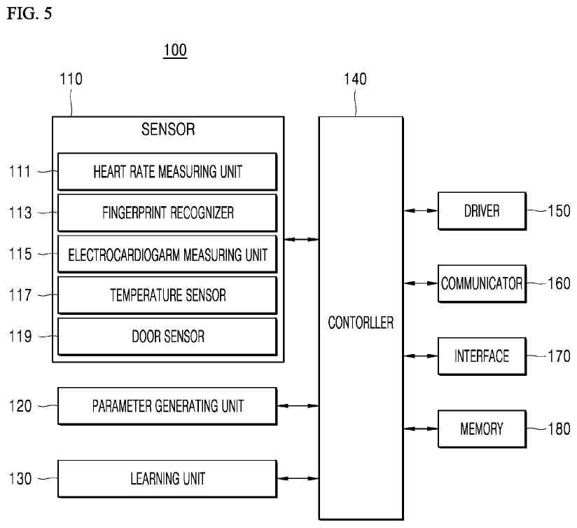

FIG. 5 is a block diagram of a home appliance according to an implementation of the present disclosure.

Referring to FIG. 5, according to an exemplary implementation of the present disclosure, a home appliance 100 includes a sensor 110, a parameter generating unit 120, a learning unit 130, a controller 140, a driver 150, a communicator 160, an interface 170, and a memory 180.

As shown in FIG. 5, the sensor 110, the parameter generating unit 120, the learning unit 130, the controller 140, the driver 150, the communicator 160, the interface 170, the memory 180 included in the home appliance 100 may be implemented as a processor. Alternatively, some of these components may be implemented as a processor. A processor may also provide processing and memory functions.

The sensor 110 includes various types of sensors. Specifically, the sensor 110 includes a heart rate measuring unit 111, a fingerprint recognizer 113, an electrocardiogram measuring unit 115, a temperature sensor 117, and a door sensor 119.

The heart rate measuring unit 111 may measure the heart rate of the user. As described above, the heart rate measuring unit 111 may include a light source transmitter 111a and a light source receiver 111b. Information on the heart rate of the user measured by the heart rate measuring unit 111 may be transmitted to the controller 140. In addition, information on the heart rate of the user measured by the heart rate measuring unit 111 may be accumulated and stored in the memory 180.

The fingerprint recognizer 113 may recognize the fingerprint of the user. Fingerprint information of the user recognized by the fingerprint recognizer 113 may be transmitted to the controller 140. The controller 140 may separately store and manage the health information value of the user (that is, the information measured by the heart rate measuring unit 111 or the electrocardiogram measuring unit 115) in a user account associated with the fingerprint information of the user. In addition, the controller 140 may store and manage a pattern of opening the door of the user in the user account associated with the fingerprint information of the user.

The electrocardiogram measuring unit 115 may measure the electrocardiogram of the user. Information on the electrocardiogram of the user measured by the electrocardiogram measuring unit 115 may be transmitted to the controller 140. In addition, the electrocardiogram of the user measured by the heart rate measuring unit 111 may be accumulated and stored in the memory 180.

A temperature sensor 117 may measure an inner temperature of the home appliance 100. The temperature sensors 117 are provided in the respective storage spaces defined by the home appliance 100 and may measure the temperatures of the respective storage spaces separately. The temperature information measured by the temperature sensor 117 may be transmitted to the controller 140.

The door sensor 119 may measure the state of the doors 21, 31, 41, and 51 provided in the home appliance 100. The door sensors 119 may be provided for doors 21, 31, 41, and 51.

The door sensor 119 may sense whether the doors 21, 31, 41, 51 are open or closed. Further, the door sensor 119 may generate a pattern of opening the door including information on state of opening the doors 21, 31, 41, and 51 for each period of time. For example, the pattern of opening the door may include operation information indicating changes of a state of the door and time information on occurrence of the change of the state of the door.

The generated pattern of opening the door may be transmitted to the learning unit 130, the controller 140, and the memory 180.

Although not clearly shown in the figures, the sensor 110 may further include a piezoelectric sensor. At this time, the piezoelectric sensor may sense whether the hand of the user contacts the home appliance 100. The signal measured by the piezoelectric sensor may be transmitted to the controller 140.

The controller 140 may provide the other components included in the sensor 110 (that is, the heart rate measuring unit 111, the fingerprint recognizer 113, the electrocardiogram measuring unit 115, the temperature sensor 117, and the door sensor 119) only when the predetermined pressure is sensed by the piezoelectric sensor, thereby minimizing the amount of power used by each sensor 110.

The parameter generating unit 120 may generate a parameter based on the temperature information measured by the sensor 110. The parameter generating unit 120 may accumulate information on the sensed values (that is, information on an inner temperature) in the memory 180, and store the sensed values, and generate a plurality of parameters.

The parameter generating unit 120 may generate the parameter based on a pattern of opening the door generated by the door sensor 119 and stored in the memory 180.

The parameter generated by the parameter generating unit 120 may include a current pattern of opening the door, a past pattern of opening the door on each day same as the current day, and a past pattern of opening the door for a predetermined period of time before a current date.

For example, when a current date is Saturday, July 30, the parameter generating unit 120 may extract the current pattern of opening the door, the pattern of opening the door on past Saturdays, and the pattern of opening the door for three weeks before the current date as a parameter. However, this is merely an example, and the parameters generated by the parameter generating unit 120 are not limited thereto.

The parameter generated by the parameter generating unit 120 may be provided to the learning unit 130 as a learning factor.

The learning unit 130 receives the parameter from the parameter generating unit 120 as a learning factor and outputs a matching rate of the pattern of opening the doors as an output thereof. The matching rate represents a degree of matching between the current pattern of opening the door and the past pattern of opening the door.

Specifically, the learning unit 130 may receive parameters (learning factors) generated by the parameter generating unit 120. Then, the learning unit 130 may apply the received parameter to a structure of deep learning in the learning unit 130 to calculate the matching rate of the past pattern of opening the door with the current pattern of opening the door. At this time, the learning unit 130 may estimate the matching rate of the past pattern of opening the door with the current pattern of opening the door using a predetermined learning algorithm input in advance.

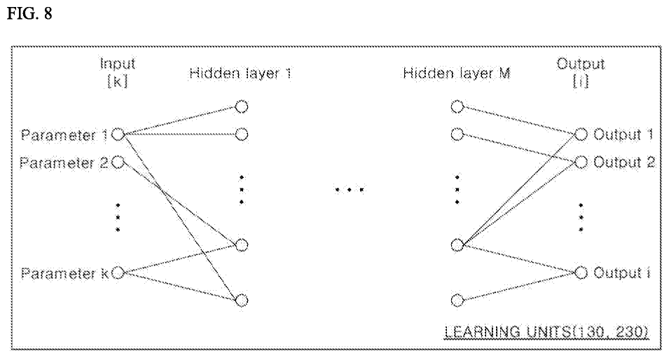

The matching rate of the past pattern of opening the door with the current pattern of opening the door may be determined based on the output information of the learning unit 130 that calculates the current state based on the machine learning. A detailed description of the learning unit 130 will be described below with reference to FIG. 8.

The controller 140 may perform overall control of the components included in the home appliance 100.

The controller 140 receives information on the matching rate of the past pattern of opening the door with the current pattern of opening the door from the learning unit 130. Then, the controller 140 compares the received matching rate with a predetermined reference matching ratio. When the matching rate is less than the predetermined reference matching rate, the controller 140 may generate a first message with respect to the emergency situation and may transmit the first message to the user terminal 300 registered in advance.

For example, the first message may include information that ‘symptom of an abnormality in a current use pattern of the user with respect to the refrigerator has occurred’. However, this is only an example, and the present disclosure is not limited thereto.

On the other hand, the controller 140 receives the health information value of the user from the sensor 110. Then, the controller 140 calculates an average of electrocardiogram and a heart rate based on the health information value that is accumulated and stored in the memory 180. At this time, the controller 140 may calculate an average of the electrocardiogram and the heart rate of the user for a predetermined period of time (for example, for one month). Then, the controller 140 compares the calculated average of the electrocardiogram and the heart rate with the current electrocardiogram and heart rate.

At this time, when the difference between the average of the electrocardiogram and the heart rate of the user and the current electrocardiogram and heart rate of the user exceeds a range of reference error, the controller 140 may generate a second message with respect to the emergency situation and transmit the generated second message to the user terminal 300 registered in advance.

For example, the second message may include information that “symptom of abnormality in health of the user is generated”. However, this is only one example, but the present disclosure is not limited thereto.

Meanwhile, the controller 140 receives fingerprint information of the user from the fingerprint recognizer 113. Then, the controller 140 may log-in the user account related to the received fingerprint information. Then, after the log-in, the data received from the sensor 110 may be separately stored in a particular section of the memory 180 corresponding to the user account.

As a result, the controller 140 may separately manage the pattern of opening the door and the health information value with respect to a plurality of users. Thus, a home appliance 100 may provide the plurality of users with the same service.

The driver 150 may adjust the inner temperature of the home appliance 100. The driving principle for the cooling and heat circulation of the driver 150 is disclosed in detail in the known documents in the related art, and a detailed description will be omitted below. The operation of the driver 150 may be controlled by the controller 140.

The communicator 160 includes one or more communication modules. Accordingly, the communicator 160 may perform various kinds of wireless communication with other electronic devices and exchange various signals. For example, the communicator 160 may exchange data with other servers or user terminals using a wireless Internet network.

The interface 170 may include a plurality of operation buttons and a display. The display may display information on command input by a user, a processing result in response to a command input by the user, an operation state, an error state, and the like of the refrigerator. In addition, the interface 170 may have a touch pad and a touch screen, which are coupled to each other.

The memory 180 records various kinds of information necessary for the operation of the home appliance, and may include a volatile or non-volatile recording medium.

Specifically, data on the pattern of opening the door used to generate the parameter may be stored in the memory 180 in the form of a database. The information on the electrocardiogram or the heart rate measured by the sensor 110 may be stored in the memory 180 in the form of a database. In addition, information on each user account, fingerprint information and personal information connected to the user account may be stored in the memory 180 in the form of a database.

The learning unit 190 may perform the machine learning with respect to the parameters input as a learning factor. The memory 180 may store data used for the machine learning, result data, and the like.

In more detail, technology of deep learning, which is a kind of machine learning that implements artificial intelligence, denotes learning with a deep level, in multi steps, based on the data.

The deep learning may represent a set of machine learning algorithms that may be used to extract key data from a large amount of data as it goes higher step.

A structure of the deep learning may include an artificial neural network (ANN). For example, the structure of the deep learning may include a deep neural network (DNN) such as a convolutional neural network (CNN), a recurrent neural network (RNN), and a deep belief network (DBN).

The learning unit 130 may use various types of known structures of deep learning. For example, the learning unit 190 may use a structure such as the convolutional neural network (CNN), the recurrent neural network (RNN), and the deep belief network (DBN).

Specifically, the convolutional neural network (CNN) is a model that simulates a function of brain of a person made based on an assumption that basic features of an object are extracted, when the person recognizes the object, and complex calculation is performed in the brain and the object is recognized based on the result thereof.

Recurrent neural network (RNN) is widely used for natural language processing, and the like, and may have a structure that is effective for processing time-series data that is changed over time and may have a structure of the ANN by stacking layers every time.

The deep belief network (DBN) has a structure of deep learning that is made by stacking restricted boltzman machine (RBM) which is a deep learning technique with many layers. When the learning of the restricted boltzman machine (RBM) is repeated and the predetermined number of layers is formed, the deep belief network (DBN) that has the corresponding number of layers may be provided.

Meanwhile, the learning based on the ANN of the learning unit 190 may be performed by adjusting the weight of an inter-node connection line (adjusting a bias value as necessary) so that a desired output is obtained for a given input.

Further, the ANN may enable continuously updating a weight value by learning. Back propagation, and the like may be used to perform the learning based on the ANN.

Meanwhile, the memory 180 may have an artificial neural network previously learned by machine learning.

That is, according to an implementation of the present disclosure, the home appliance 100 may perform determining the situation based on the machine learning with the received pattern of opening the door as the input data. At this time, both unsupervised learning and supervised learning may be used as a method of machine learning based on the ANN. In addition, the learning unit 130 may update the structure of the ANN that enables calculates the matching rate of the past pattern of opening the door with the current pattern of opening the door, after learning, according to the setting.

Additionally, according to another implementation of the present disclosure, the operation of the learning unit 130 may be performed by a separate cloud server 200. According to an implementation of the present disclosure, a system in which an operation of machine learning is performed inside thereof such as the home appliance 100 is referred to as ‘an internal learning-based system’.

On the other hand, a system in which the operation of the machine learning is performed by an external server (for example, the cloud server 200) is referred to as ‘an external learning-based system’.

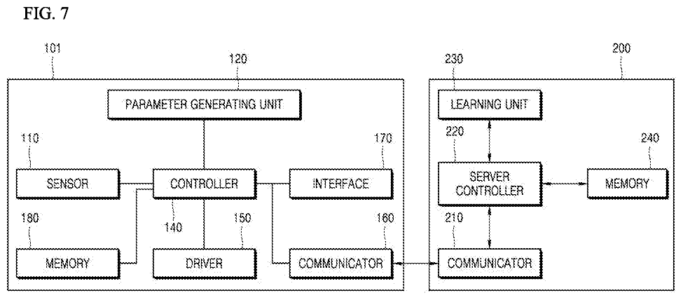

Hereinafter, the home appliance 101 and the cloud server 200 that constitute the external learning-based system will be described with reference to FIGS. 6 and 7.

FIG. 6 is a schematic view of a home appliance and a cloud server according to another implementation of the present disclosure. FIG. 7 is a block diagram of relations between the home appliance and the cloud server of FIG. 6. Hereinafter, descriptions overlapping with those described above will be omitted.

Referring to FIGS. 6 and 7, among the components of the home appliance 101 that has the external learning based system, the sensor 110, the parameter generating unit 120, the controller 140, the driver 150, the communicator 160, the interface 170, and the memory 180 may operate substantially the same as the respective components of the home appliance 100 described above with reference to FIG. 5. The description of the components overlapping with the above-described home appliance 100 will be omitted.

The parameter generating unit 120 may generate one or more parameters based on the pattern of opening the door stored in the memory 180. The parameter generating unit 120 may store the pattern of opening the door sensed by the sensor 110 in the memory 180 for predetermined period of time and generate a plurality of parameters based on the stored pattern of opening the door.

For example, the plurality of parameters generated by the parameter generating unit 120 may include a current pattern of opening the door, a past pattern of opening the door on each day same as the current day, and a past pattern of opening the door during a predetermined period of time before the current date.

Then, the controller 140 transmits the generated parameter to the cloud server 200 through the communicator 160.

The cloud server 200 includes a communicator 210, a server controller 220, and a learning unit 230.

The communicator 210 wirelessly communicates with the communicator 160 of the home appliance 100 to exchange data.

The server controller 220 transmits the parameters received from the communicator 210 to the learning unit 230.

The learning unit 230 receives the parameter as a learning factor (i.e., an input factor). Then, the learning unit 130 may output the matching rate between the current pattern of opening the door and the past pattern of opening the door as output factors.

At this time, the learning unit 230 may generate links between one or more hidden layers and each input/output factor, and bias or the weight of each link in a learning process, and may store information updated from the outside. In this case, the learning unit 230 may be stored in the cloud server 200 in different versions.

In summary, the cloud server 200 may receive information on a learning factor (i.e., a parameter) from the home appliance 101 and generate an output factor corresponding thereto. In addition, the learning factors provided by the home appliance 101 may be continuously input to the learning unit 230 to update the learning unit 230. The learning unit 230 may estimate level of load using a predetermined learning algorithm.

Then, the server controller 220 compares the matching rate output by the learning unit 230 with a predetermined reference matching rate.

Then, when the output matching rate is greater than the reference matching rate, the server controller 220 may determine that an emergency situation occurs to the user.

When it is determined that the emergency situation occurs, the server controller 220 may transmit a message with respect to the emergency situation to the user terminal 300.

In addition, the cloud server 200 receives health information value on the heart rate and electrocardiogram of the user measured by the sensor 110 of the home appliance 101. The received health information value is stored in the memory 240 of the cloud server 200.

For reference, the cloud server 200 may receive the fingerprint information of the user and may store the received health information value in a user account connected to the received fingerprint information. Similarly, the cloud server 200 may store the received pattern of opening the door in the corresponding user account. Accordingly, the cloud server 200 may separately manage the health information value or the pattern of opening the door of a plurality of users, using respective user accounts.

Then, the server controller 220 calculates an average of the electrocardiogram and heart rate based on the health information value that is accumulated and stored in the memory 240. At this time, the server controller 220 may calculate an average of the electrocardiogram and heart rate based on the health information value of the user for a predetermined period of time (for example, for one month). Then, the server controller 220 compares the calculated average of the electrocardiogram and heart rate with the current electrocardiogram and heart rate.

At this time, when the difference between the average of the electrocardiogram and heart rate and the current electrocardiogram and heart rate exceeds the range of the reference error, the server controller 220 may generate a message with respect to the emergency situation and transmit the message to the user terminal 300 registered in advance.

That is, the present disclosure may enable quickly responding to an accident when the accident occurs to the user by sending a message with respect to an emergency situation to the user terminal 300 registered in advance.

In addition, the present disclosure may enable improving accuracy of determining whether an emergency situation occurs based on both the use pattern of the user with respect to the home appliance 101 and the health information value of the user.

Hereinafter, configurations of the learning units 130 and 230 according to some implementations of the present disclosure will be described.

FIG. 8 shows a configuration of a learning unit according to some implementations of the present disclosure.

Referring to FIG. 5, learning units 190 and 230 include an input layer that has N-number of parameters as input nodes, an output layer (output) that has a matching rate of a current pattern of opening a door and a past pattern of opening a door as an output node, and M-number of hidden layers between the input layer and the output layer.

The parameter may include a current pattern of opening the door, a past pattern of opening the door on the same date as the current pattern of opening the door, and a past pattern of opening the door for a predetermined period of time before a current date.

A weight of the edge that connects the nodes of the layers may be set. The weight thereof or edge may be added, removed, or updated during the learning process. Thus, through the learning process, the weights of the edges and the nodes arranged between k-number of input nodes and i-number of output nodes may be updated.

As shown in FIG. 8, i-number of output nodes may be arranged so as to output values such as 1/0 or probability for each mode. For example, the output node may be a matching rate of the current pattern of opening the door and the past pattern of opening the door. Further, the output node may have a steady-state probability and an abnormal state probability (that is, two kinds of probabilities). However, these are merely some examples, and the output node may be modified variously and implemented.

All nodes and edges may be set to initial values before the learning units 190 and 230 perform the learning. However, when the information is accumulatively input, the weights of the nodes and the edges are changed, and matching of parameters input as learning factors (that is, the current pattern of opening the door and the past pattern of opening the door) with a value allocated to the output node (that is, the matching rate of the past pattern of opening the door with the current pattern of opening the door) may be made.

In addition, when the cloud server 200 is used, the learning unit 230 may receive information on a large number of parameters and process the parameters. Therefore, the learning unit 230 may perform the learning based on a large amount of data.

In summary, the weights of nodes and edges between the input node and the output node included in the learning units 130 and 230 of FIG. 8 may be updated by a learning process of the learning units 130 and 230. The pattern matching rate output by the learning units 130 and 230 may be used to determine whether the home appliance 100 is in an emergency situation.

Hereinafter, a method for operating the home appliance according to some implementations of the present disclosure will be described in detail.

Hereinafter, for convenience of explanation, according to an implementation of the present disclosure, the home appliance 100 that operates based on an internal learning-based system will be described.

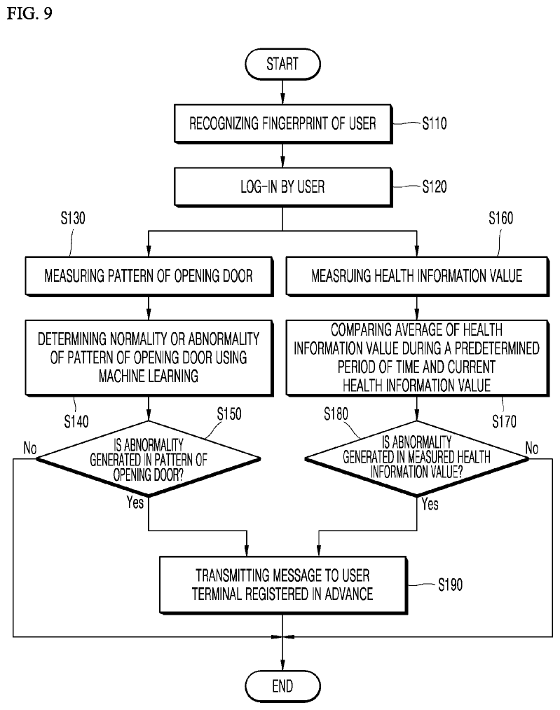

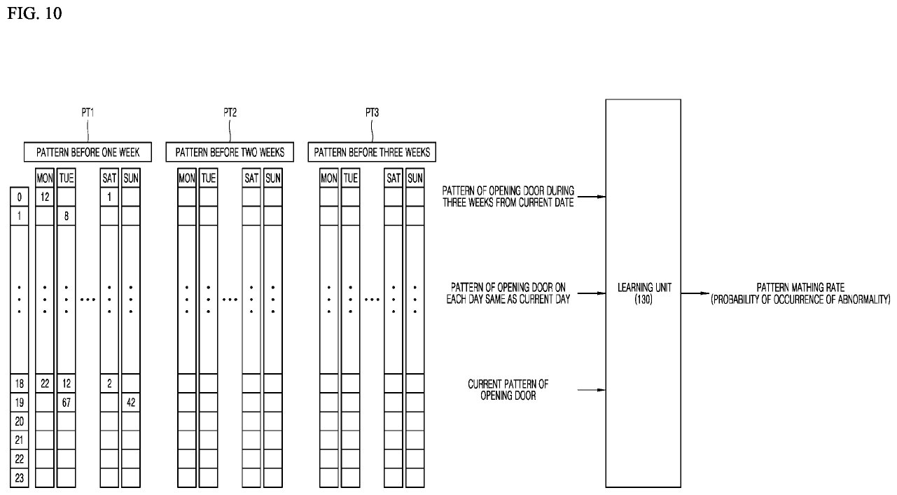

FIG. 9 is a flowchart of a method for operating a home appliance according to some implementations of the present disclosure. FIG. 10 shows a method for determining whether there is an abnormality in the pattern of opening the door in S140 of FIG. 9.

Referring to FIG. 9, according to some implementations of the present disclosure, in the method of operating a home appliance, a fingerprint recognizer 113 recognizes a fingerprint of a user first (S110). The information on the recognized fingerprint is transmitted to a controller 140.

Then, the controller 140 logs-in a user account connected to the received fingerprint information (S120). Accordingly, the controller 140 may access the data on the past health information value and pattern of opening the door of the user stored in the user account.

Then, a door sensor 119 measures the pattern of opening the door of the user (S130). Information on the measured pattern of opening the door may be stored in a memory 180.

Then, the parameter generating unit 120 may generate parameters based on the pattern of opening the doors stored and accumulated in the memory 180. At this time, the parameter may include a current pattern of opening the door, a past pattern of opening the door for the same date as the current pattern of opening the door, and a past pattern of opening the door for a predetermined period of time before a current date.

For example, referring to FIG. 10, the memory 180 may store the pattern of opening the door three weeks ago from the current date. Each past pattern of opening the door may be stored on a weekly basis. That is, a user terminal 300 may store a pattern of opening the door before one week PT1, a pattern of opening the door before two weeks PT2, and a pattern of opening the door before three weeks PT3.

Then, information on the generated parameter may be transmitted to the learning unit 130.

That is, the patterns of opening the doors PT1, PT2, and PT3 for three weeks before the current date may be input to the learning unit 130 as a first parameter.

In addition, the pattern of opening the door on each day same as the current day may be input to the learning unit 130 as a second parameter. That is, when the current day is Tuesday, a pattern of opening the door on every Tuesday for three weeks before the current date may be input to the learning unit 130 as the second parameter.

Further, the current pattern of opening the door may be input to the learning unit 130 as the third parameter. In summary, the first to third parameters may be applied to the input node of the learning unit 130.

Then, the learning unit 130 may receive the parameter as a learning factor and may output the matching rate between the current pattern of opening the door and the past pattern of opening the door as an output to the learning parameter. Information on the output matching ratio is transmitted to the controller 140.

Then, the controller 140 determines whether the pattern of opening the door is abnormal based on the received matching rate (S140). At this time, the controller 140 compares the matching rate output by the learning unit 230 with a predetermined reference matching rate.

If the output matching rate is greater than the reference matching rate, the controller 140 may determine that the emergency situation occurs to the user (S150). That is, when the pattern of opening the door of the user differs from a general pattern of opening the door, the controller 140 may determine that the emergency situation occurs to the user.

Then, when the abnormality is found in the pattern of opening the door of the user, the controller 140 may transmit the first message with respect to the emergency situation to the user terminal 300 registered in advance (S190). The first message may include information that ‘symptom of an abnormality occurs in a use pattern of the user with respect to the refrigerator’. However, this is only an example, and the present disclosure is not limited thereto.

Meanwhile, the sensor 110 may measure the health information value of the user (S160). Specifically, the heart rate measuring unit 111 may measure the heart rate of the user. In addition, the electrocardiogram measuring unit 115 may measure the electrocardiogram of the user. Information on the measured heart rate and electrocardiogram may be stored in the memory 180.

Then, the controller 140 calculates an average of the electrocardiogram and the heart rate based on the health information value that is accumulated and stored in the memory 180. For example, the controller 140 may calculate the average of the electrocardiogram and the heart rate for one month.

Then, the controller 140 compares the calculated average of the electrocardiogram and the heart rate and the current electrocardiogram and heart rate (S170).

Then, based on whether a difference between the average of the electrocardiogram and the heart rate and the current electrocardiogram and heart rate exceeds the range of reference error, the controller 140 determines whether an abnormality occurs in the electrocardiogram and the heart rate based on the health information value of the user (S180).

Then, when it is determined that the abnormality occurs based on the health information value of the user, the controller 140 may generate a second message with respect to the emergency situation and may transmit the second message to the user terminal 300 (S190). The second message may include information that ‘symptom of an abnormality occurs in the health of the user’. However, this is only an example, and the present disclosure is not limited thereto.

Accordingly, the present disclosure may enable determining whether the emergency situation occurs by comparing a general pattern of opening the door of a user with a recent pattern of opening the door. At this time, the present disclosure may enable improving the accuracy of determining the emergency situation using the machine learning.

In addition, the present disclosure enables measuring the health information value of the user by the sensor provided on the handle of the home appliance, thereby reducing the effects of the user to visit the hospital for healthcare. In addition, the user may detect the symptom of the disease early by periodically managing the normality or abnormality of the health of the user.

That is, according to the present disclosure, both the use pattern of the user with respect to the home appliance and the health information value of the user may be used to improve the accuracy of determining whether the emergency situation occurs.

Therefore, the present disclosure may enable quickly responding to the accident when the accident occurs to the user as one person household. Thus, the reliability of the user with respect to the product may be improved. The brand awareness of the product may also be improved.

While the present disclosure has been mainly described with reference to the implementation of the present disclosure hereinabove, various modifications and changes may be made at the level of those skilled in the art. Therefore, unless such modifications and changes do not deviate the scope of the present disclosure, it will understand that they are included in the scope of the present disclosure.

### DESCRIPTION OF REFERENCE NUMERALS

| | |
|---|---|
| 100: Home appliance | 110: Sensor |
| 120: Parameter generating unit | 130: Learning unit |
| 140: Controller | 150: Driver |
| 160: Communicator | 170: Interface |
| 180: Memory | 200: Cloud server |
| 210: Communicator | 220: Server controller |
| 230: Learning unit | 300: User terminal |

What is claimed is:

1. A home appliance, comprising:
a door with a handle;
a heart rate measuring unit provided at the handle and configured to measure a heart rate of a user;
an electrocardiogram measuring unit provided at the handle and configured to measure an electrocardiogram of the user;
a memory configured to store information; and
one or more controllers configured to:
store, in the memory, measured user health information values comprising the measured electrocardiogram and the measured heart rate of the user;
compare average values of user health information values stored in the memory over a predetermined time period with the measured user health information values; and
generate a message A indicating an emergency situation and transmit the generated message A to a user terminal based on a difference between the average values of user health information values and the measured user health information values exceeding a predetermined range.

2. The home appliance of claim 1, further comprising a door sensor configured to sense an open state or closed state of the door,
wherein the one or more controllers are further configured to:
determine a pattern of open and closed states of the door and store the determined pattern in the memory;
generate a message B indicating an emergency situation and transmit the generated message B to the user terminal based on an output value of a learning model being less than a predetermined threshold matching rate,
wherein, an input value of the learning model is one or more parameters based on stored patterns of open and closed states of the door previously stored in the memory, and the output value of the learning model is a matching rate between a current pattern of open and closed states of the door and the stored pattern.

3. The home appliance of claim 2, wherein the one or more parameters comprises the current pattern of open and closed states of the door, a past pattern of open and closed states of the door on each previous same day of the week as a current day, and a past pattern of open and closed states of the door for a predetermined time period before a current date.

4. The home appliance of claim 2, wherein the learning model comprises:
an input layer configured to receive the one or more parameters as inputs;
an output layer configured to output the matching rate; and
at least one hidden layer between the input layer and the output layer,
wherein weights of nodes and edges between the input node and the output node are updated by a learning process of the learning model.

5. The home appliance of claim 2, wherein the first or second message comprises information indicating the emergency situation related to user health information values or a pattern of open and closed states of the door.

6. The home appliance of claim 2, further comprising a fingerprint sensor provided at the handle,
wherein the one or more controllers are further configured to:
recognize a fingerprint sensed by the fingerprint sensor; and
store the measured health information values of the user and the determined pattern of open and closed states of the door in connection with a user account associated with the recognized fingerprint.

7. The home appliance of claim 1,
wherein the heart rate measuring unit comprises a light source transmitter configured to emit a light of a predetermined magnitude and a light source receiver configured to receive the emitted light, and
wherein the heart rate of the user is measured based on changes in amount of the emitted light received at the light source receiver.

8. The home appliance of claim 1, wherein the electrocardiogram measuring unit comprises a first measuring unit arranged at an outer surface of the door and a second measuring unit arranged at an outer surface of another door of the home appliance.

9. A cloud server communicating with a home appliance, comprising:
a communicator; and
one or more server controllers configured to:
receive a parameter from the home appliance via the communicator, wherein the received parameter is based on a current pattern of open and closed states of the door determined by the home appliance; input the received parameter to a learning model which outputs a matching rate between the current pattern of open and closed states of the door and a stored pattern of open and closed states stored in a memory;
generate a message B indicating an emergency situation when the matching rate is less than a predetermined reference matching rate; and
transmit the generated message to a user terminal.

10. The cloud server of claim 9, wherein the parameter comprises the current pattern of open and closed states of the door, a past pattern of open and closed states of the door on each previous same day of the week as a current date, and a past pattern of open and closed states of the door for a predetermined time period before the current date.

11. The cloud server of claim 9, wherein the learning model comprises:
an input layer configured to receive the parameter as an input;
an output layer configured to output the matching rate; and
at least one hidden layer between the input layer and the output layer,
wherein weights of nodes and edges between the input node and the output node are updated by a learning process of the learning model.

12. The cloud server of claim 9, further comprising a memory,
wherein the one or more server controllers are further configured to:
receive, from the home appliance via the communicator, health information values comprising electrocardiogram information of a user measured by an electrocardiogram measuring unit and heart rate information of the user measured by a heart rate measuring unit;
compare average electrocardiogram values stored in the memory and average heart rate values stored in the memory over a predetermined time period with the received electrocardiogram information and heart rate information of the user.

13. The cloud server of claim 12, wherein the one or more server controllers are further configured to generate a second message indicating an emergency situation and transmit the generated a message A to the user terminal based on a difference between the average electrocardiogram and heart rate values and the received electrocardiogram and heart rate information of the user exceeding a predetermined range.

14. The cloud server of claim 12, wherein the one or more server controllers are further configured to:
receive, from the home appliance via the communicator, fingerprint information of the user; and
store the received health information values of the user and the pattern of open and closed states of the door received from the home appliance in connection with a user account associated with the fingerprint information of the user.

15. A home appliance, comprising:
a door with a handle;
a heart rate measuring unit provided at the handle and configured to measure a heart rate of a user;
an electrocardiogram measuring unit provided at the handle and configured to measure an electrocardiogram of the user;
a memory configured to store information; and
one or more processors configured to:
store, in the memory, measured health information values comprising the measured electrocardiogram and the measured heart rate of the user;
compare average values of user health information values stored in the memory over a predetermined time period with the measured health information values; and
generate a t message A indicating an emergency situation and transmit the generated message A to a user terminal based on a difference between the average values of user health information values and the measured user health information values exceeding a predetermined range.

16. The home appliance of claim 15, further comprising a door sensor configured to sense an open state or closed state of the door,
wherein the one or more processors are further configured to:
determine a pattern of open and closed states of the door and store the determined pattern in the memory;
generate a message B indicating an emergency situation and transmit the generated message B to the user terminal based on an output value of a learning model being less than a predetermined threshold matching rate,
wherein, an input value of the learning model is one or more parameters based on stored patterns of open and closed states of the door previously stored in the memory, and the output value of the learning model is a matching rate between a current pattern of open and closed states of the door and the stored pattern.

17. The home appliance of claim 16, wherein the one or more parameters comprises the current pattern of open and closed states of the door, a past pattern of open and closed states of the door on each previous same day of the week as same as a current day, and a past pattern of open and closed states of the door for a predetermined time period before a current date.

18. The home appliance of claim 16, wherein the learning model comprises:
an input layer configured to receive the one or more parameters as;
an output layer configured to output the matching rate; and
at least one hidden layer between the input layer and the output layer,
wherein weights of nodes and edges between the input node and the output node are updated by a learning process of the learning model.

19. The home appliance of claim 16, further comprising a fingerprint sensor provided at the handle,
wherein the one or more processors are further configured to:
recognize a fingerprint sensed by the fingerprint sensor; and
store the measured health information values of the user and the determined pattern of open and closed states of the door in connection with a user account associated with the recognized fingerprint.

20. The home appliance of claim 15,
wherein the heart rate measuring unit comprises a light source transmitter configured to emit a light of a predetermined magnitude and a light source receiver configured to receive the emitted light, and
wherein the heart rate of the user is measured based on a change in amount of the emitted light received at the light source receiver.
